(19) United States
(12) Patent Application Publication
(10) Pub. No.: US 2023/0210756 A1
(43) Pub. Date: Jul. 6, 2023

(54) Tattoo Towelettes

(71) Applicant: Megan Herzberg, Nassau (BS)

(72) Inventor: Megan Herzberg, Nassau (BS)

(21) Appl. No.: 17/567,065

(22) Filed: Dec. 31, 2021

(51) Int. Cl. A61K 8/92; A61Q 1/00; A61K 8/02; A61Q 19/10; A45D 34/04

(52) U.S. Cl. CPC A61K 8/92; A61Q 1/00; A61K 8/0208; A61Q 19/10; A45D 34/04; A61K 2800/56; A45D 2200/1027

(57) ABSTRACT

A moisturing towelette is impregnated with a liquid formulation that, when applied to the skin, brightens colors of a tattoo disposed beneath the epidermis, while cleaning the tattoo and care for the skin.

### CROSS-REFERENCES TO RELATED APPLICATIONS

None

### FIELD

The technology herein relates to tattoo care, and more particularly to portable wipes or towelettes for cleaning, caring and brightening a tattoo at the same time.

### BACKGROUND & SUMMARY

Tattoo needles deposit ink beneath the epidermis. See FIG. 1. This is deep enough that the ink won't be immediately shed with the skin cells of the upper layers. Macrophage cells (a kind of white blood cell from the body's immune system) will treat the injected pigment as a wound and try to absorb the pigment and remove it from the site of the wound. The pigment in the ink is too large for the macrophages to remove, so the macrophages—with the ink inside—end up getting stuck in the dermis.

As a tattoo ages, ink particles move into the deeper dermis over time due to the action of mobile phagocytic cells, causing the tattoo to look bluish, faded and blurry. The ultraviolet UV frequencies in sunlight are energetic enough to break down the ink molecules over time, so tattoos that are often exposed to bright sunlight—such as on the fingers or face—fade quicker than those covered by clothing. See Villazon, “Why Do Tattoos Fade?”, Science Focus (BBC 2021). https://www.sciencefocus.com/the-human-body/why-do-tattoos-fade/

Tattoo owners sometimes apply oils, moisturizers, serums or balms on their skin to brighten the appearance of their tattoos or make them “pop.” A number of lotions and balms have been developed for brightening tattoos. See for example:

US20160287288 discloses a tattoo brightener serum comprising one or more of the group comprising Isododecane, Phenyl Trimethicone, Isostearyl Palmitate,

Diethicone, oxidized corn oil, Tocopheryl Acetate (vitamin E), Glycine Soja (soybean) seed extract, Ceramides (Sphigolipids), Cyclocopentasiloxane, Dimethiconol, C12-15, Alkyl Benozoate, Olea Europea, Fruit Oil, Fragrance, Argania Spinosa Kernal Oil, Helanthus Annuus (Sunflower) Seed Oil, Passiflora Edulis Seed Oil, Copaifera Officinals (Balsam Copaiba) Resin, Chloroxylenol, Avobenzone, Octinoxate, Octisalate, Octorcrylene, Oxybenzone, Paraffin, Tocopherol, Mineral Oil, Lanolin, Dimethicone, and Beeswax. However, this serum is used in conjunction with an abrasive that abrades the outer surface of the skin, which might have detrimental effects.

Another example tattoo serum for brightening comprises peptides, hyaluronic acid, amino acids, antioxidants, vitamins, and phospholipids.

Another example tattoo brightener is a balm comprising Butyrospermum Parkii (Shea Butter), Orbignya Oleifera (Babassu) Oil, Cera Alba (Yellow) Bees Wax, Vitis Vinifera (Grape) Seed Oil, 100% Pure Dudeness, Cetyl Alcohol, d-Alpha Tocopherol, and Rosmarinus Officinalis (Rosemary) Leaf Extract.

Another example tattoo brightener is a balm comprising shea butter, cocoa butter, beeswax, calendula, sweet almond, lavender, frankincense, and cucumber.

Such balm formulations are not liquid at room temperature, which allows smearing of the balm on to the skin without risk of spillage. However, balms can be inconvenient to apply, especially during a busy night out or on the beach or in hot weather, taking into account that balms are getting liquid when they experience heat. Further improvements are desirable.

### BRIEF DESCRIPTION OF THE DRAWINGS

FIG. 1 shows an example tattooing process in which the tattoo needles deposit ink beneath the epidermis.

FIG. 2 shows example packaging.

### DETAILED DESCRIPTION OF EXAMPLE NON-LIMITING EMBODIMENTS

In one embodiment, special tattoo wipes are made of biodegradable microfine tissue with a gentle care, clean and brighten formulation suitable for all kinds of tattoos.

The technology herein provides a piece of a special tattoo care, cleaning and brightening cloth, towelette or wipe. It consists of 3 special parts.
1. The packaging
2. The cloth, towelette or wipe
3. Special brightening fluid or liquid in the cloth, towelette or wipe.

Packaging

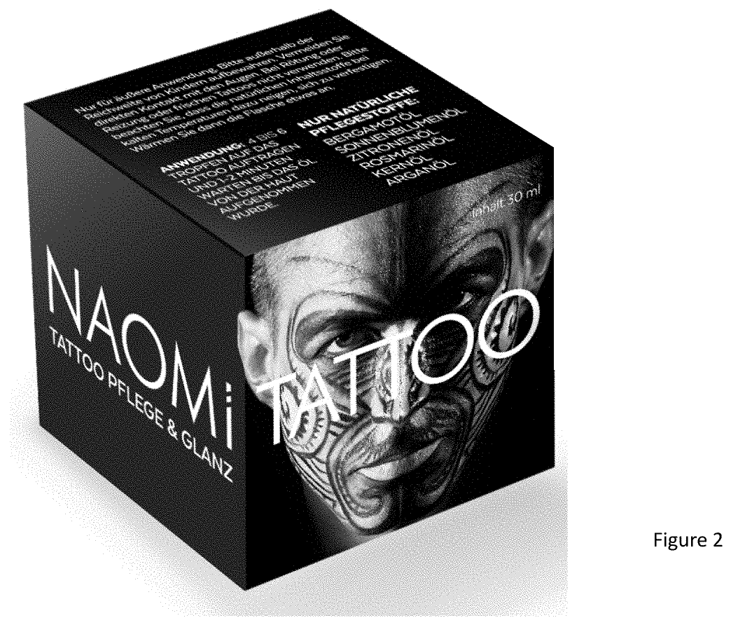

One embodiment uses a coated packaging (see FIG. 2) that is capable of keeping the contents sealed and is resistant to oily liquids. Packaging can be designed as single use or can be constructed as a pouch that can be sealed by the user for multiple use. This means that this packaging also doubles as a reusable waterproof travel pouch. This packaging will cover the “how to use instructions” and will be produced in this format.

Example form factor for packaging: L 110×T 185×H 60 mm

The packaging can have different measurements and will include the cloth, towelettes or wipes which will have these measurements

L 185×T 185 mm

These measurements are exemplary only and can differ.

In one embodiment, wipes are packed in a polyethylene container with a resealable lid. The top is designed to be opened effortlessly with the gloved hand, yet preserves the remaining product.

In another embodiment, wipes are packed in a resealable polyethylene pouch.

In another embodiment, wipes are packed in a sealed single use polyethylene pouch.

Other materials can be used, too.

Cloth, Towelette or Wipes

The cloth is not dry but premoistured with a special formula that brightens a tattoo and makes it “pop” or look more vivid and stand out. The formulation and its effects are discussed below.

The towels can be woven or non-woven shop towel, rag, pad, or sheet made of cellulose, wood pulp, fabric, cotton, polyester, polyester blends, polypropylene, rayon fibers or other material formed into sheets. These sheets in one embodiment are made of microfiber (or microfibre)—synthetic fiber finer than one denier or decitex thread, having a diameter of less than ten micrometers. A strand of silk is about one denier and about a fifth of the diameter of a human hair. This cloth can also be made from special papers or 100 percent natural, plant-based cotton and as example, added bamboo fibers. Such fibers comprise giant molecules that consist of many small molecules linked together. The special cloth therefore can also be made of a piece of superfine micro fiber cloth which is added with a film of a special tattoo oil liquid. Such micro fibers can comprise split microfibers that are thinner than a human hair for example with a thickness of <75 μm or <70 μm or <65 μm or <60 μm or <55 μm or <50 μm or <45 μm or <40 μm or <35 μm or <30 μm or <25 μm or <20 μm or <15 μm or <10 μm or <5μm or <1μm or <7.5 μm or <0.75 μm or <0.5 μm or <0.4 μm or <0.3 μm or <0.2 μm or <0.1 μm or <0.05 μm. Such very thin micro fibers increases the surface area the towelette can absorb.

Brightening, Cleaning and Caring Liquid or Fluid

The special tattoo care, which is designed for cleaning and brightening a tattoo plus skincare and is the liquid on cloth disclosed herein, has not only a formula to provide skin care, it deep cleans the tattoo and at the same time it is refreshening the colors of the tattoo. It can be used to care for, moisturize and clean the skin.

The oil may be made of all natural ingredients that make sure, that the tattooed skin receives care, the tattoo is cleaned and brightened at the same time.

The oils used to impregnate the towelette sheets may comprise any or all of the following:
1. Bergamot oil
2. Sunflower seed oil
3. Lemon oil
4. Rosemary Oil
5. Pumpkin seed oil
6. Argan oil

In one embodiment, the oil is natural rather than artificial. In one embodiment, the oil is light and is a non-viscous liquid at room temperature. In one embodiment, the oil is non-toxic. The oil or oils when applied to the skin will brighten the colors of faded ink within the tattoo.

Since this is especially made for people with tattoos, it differentiates very much from other use cases of any kind. It is just for tattoos and is designed to give the user the opportunity to care, clean and brighten a tattoo kind of in between.

If you are at a party and you, as a female user, refreshen your make up or your lipstick, a special and easy to use cloth with this special oil or other formulation is the perfect addition to your habit.

This is a missing link that is very special to all people with a tattoo who do not want to carry a balm to the party, beach, theatre or elsewhere.

Just a nice sealed, easy to use cloth, that can be carried in a handbag or a pocket.

The oily cloth is protected, it can not leak in to your bag, it is handy, small and helps you to care, clean and brighten your tattoo.

The product itself is a great support for tattooed people.

It is not only a formula with a special oil consisting of all natural ingredients but especially this new use case that makes the product so special.

Our formula makes sure, that the tattoo and the tattooed skin receives 3 things at the same time:
1. Care
2. Cleaning
3. Brightness

With a balm or any other product, it is in the user's discretion how much he or she is using.

Since in one embodiment the wipes receive a certain “dose” of special oil, the user can not take too much or too less. It is always a perfect and consistent result.

The formula makes sure that the oil is applied gradually and sinks in quite gently and will be absorbed by the skin within one or two minutes. There is no excess oil left on the surface of the skin.

Apart from this, there are some advantages compared to balm.

If balm or wax gets hot, it becomes liquid quite fast. That makes it impossible get the out of the tube or jar. Apart from this you typically need a sponge or paper towels to apply this to your skin.

With example non-limiting oil moisturized wipes as described herein, the user can:
use it even on the beach or in areas with higher temperatures;
always apply the right dose to care, to clean and brighten a tattoo;
be sure not having a mess with a semi liquid wax or balm that cannot take heat;
be sure that it doesn't leak;
be sure it is hygienic; and
enjoy that you do not need a sponge or other extra applicator.

These special towels moisturized with our special oil are more hygienic because the user may use one towel at the time instead of putting a used sponge into ajar over and over, again.

Users also don't have to share their balm/wax with others, too.

The wipes do cover significantly less space in a handbag than a sealed special wipe.

### EXAMPLE I

FIG. 2 shows a box for these little oil moistured cloth pieces. The box is made of a material that is impervious to oil leaking through and provides a seal so that oil cannot leak out of the box. A quantity of towelettes are stacked within the box so that the towelettes can be pulled out one at a time.

### EXAMPLE II

Each towelette is packaged independently in a foil package (like commonly available alcohol wipes but the present formulation contains no alcohol) to retain the oil formulation impregnated in the towelette. The amount of oil impregnated in each towelette is carefully controlled to provide a consistent application over a given surface area.

### EXAMPLE III

Towelettes can have different dimensions depending on the amount of coverage area. People with a small tattoo can use a small towelette, people with larger tattoos can use a medium size towelette, and people with many tattoos over a large surface area can use a large towelette.

### EXAMPLE IV

The towelette can be impregnated with a moisturing oil formulation that contains a humectant such as glycerine, an emollient and an emulsifier. The towelette is perfect for the beach or other outdoor activities since it moisturizes and brightens the appearance of the tattoo in bright sunlight.

### EXAMPLE V

The towelette may be impregnated with one or more natural oils that exude a fragrance, and/or a fragrance agent may be added to the natural oils.

### EXAMPLE VI

To use the towelette, the user extracts the towelette from its packaging and, grasping it in the hand, unfolds the towelette if it is folded and rubs the towelette over the surface of skin under which the ink of a tattoo is disposed. The absorbency of the towelette is such that the towelette deposits a thin film of oil onto the skin's surface. After a minute or two, as the skin absorbs the oil, the tattoo within the skin brightens and appears to “pop.”

### EXAMPLE VII

Each towelette is impregnated with a metered quantity of a hypoallergenic formulations including any combination of the following:

Bergamot oil (0% or <=10% or <=20% or <=30% or <=40% or <=50% or <=60% or <=70% or <=80% or <=90% or <=100%)

Sunflower seed oil (0% or <=10% or <=20% or <=30% or <=40% or <=50% or <=60% or <=70% or <=80% or <=90% or <=100%)

Lemon oil (0% or <=10% or <=20% or <=30% or <=40% or <=50% or <=60% or <=70% or <=80% or <=90% or <=100%)

Rosemary Oil (0% or <=10% or <=20% or <=30% or <=40% or <=50% or <=60% or <=70% or <=80% or <=90% or <=100%)

Pumpkin seed oil (0% or <=10% or <=20% or <=30% or <=40% or <=50% or <=60% or <=70% or <=80% or <=90% or <=100%)

Argan oil (0% or <=10% or <=20% or <=30% or <=40% or <=50% or <=60% or <=70% or <=80% or <=90% or <=100%),

where “<=” means “less than or equal to.”

Theory of Operation

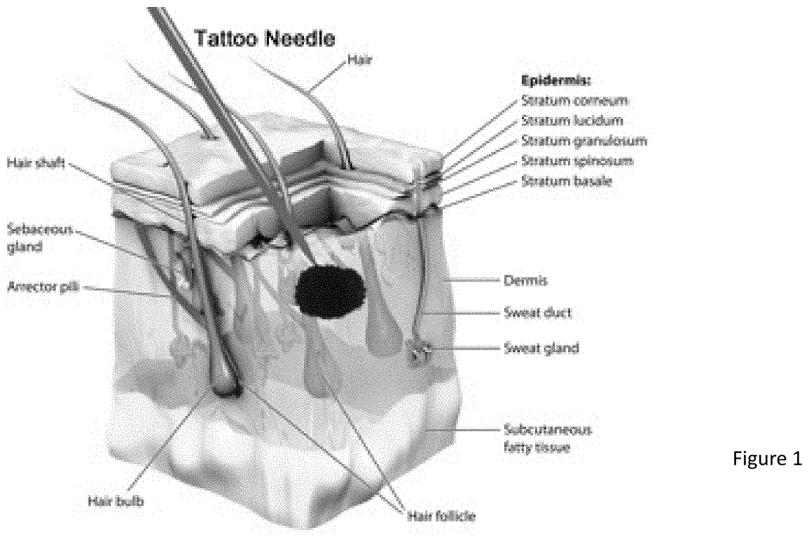

As the user rubs the towelette over the skin's surface, the towelette deposits a brightening liquid formulation on the surface of the skin. As the skin's outer surface absorbs the brightening formulation (e.g., such as through pores in the skin), the epidermis becomes more translucent or light-transmissive, enabling more clear visualization of ink stored in structures such as microphages, keratinocytes and dermal fibroblasts within the dermis beneath a layer of fibrous tissue at the epithelial-dermal junction. Oils from the towelette penetrate the epidermis through pores and the intracellular matrix of the dermis, increasing light transmissivity through the epidermis so the ink and ink-containing structures within the dermis become more visible through the epidermis. It is believed the brightening formulation substantially reduces the light scattering at the stratum corneum (see FIG. 1), corresponding to an increase of >5% or >10% or >15% or >20% or >25% or >30% or >35% or >40% or >45% or >50% or >55% or >60% or >65% or >70% or >75% or >80% or >85% or >90% or >95% or >100% or >125% or >150% or >175% or >200% or greater than 2 times or greater than 3 times or greater than 4 times or greater than 5 times in light transmissivity through the epidermis to the deeper layers of the skin (i.e., the dermis) which is where the tattoo pigmentation is located. See e.g., Jiang et al, “Appearance Benefits Of Skin Moisturization”, Skin Res Technol 2011 Feb; 17(1):51-5. doi: 10.1111/j.1600-0846.2010.00462.x. Epub 2010 Aug. 16; Lister et al, Optical Properties Of Human Skin, J. of Biomedical Optics, 17(9), 090901 (2012), https://doi.org/10.1117/1.JBO.17.9.090901; Qassem M et al, Use of reflectance near-infrared spectroscopy to investigate the effects of daily moisturizer application on skin optical response and barrier function, J Biomed Opt. 2014 Aug; 19(8):087007. doi: 10.1117/1.JBO.19.8.087007 PMID: 25166473; Crowther JM et al, Measuring the effects of topical moisturizers on changes in stratum corneum thickness, water gradients and hydration in vivo, Br J Dermatol. 2008 Sep;159(3):567-77. doi: 10.1111/j.1365-2133.2008.08703.x. Epub 2008 Jul. 4. PMID: 18616783 Clinical Trial.

Skin translucency thus increases as the skin becomes moisturized. Increased translucency of the skin layers above the deeper locations of the tattoo pigmentation by application of a moisturizing fluid by a towelette causes the tattoo pigmentation colors to appear more saturated and makes the tattoo's appearance “pop.” The viscosity and volatility of the liquid formulation used to impregnate the towelettes are selected so the towelette deposits a sufficient amount of liquid formulation onto the outer surface of the skin to remain on the skin for a sufficient amount of time (e.g., 60 to 120 minutes) without evaporating to be absorbed by the epidermis and substantially increase light-transmissivity of the epidermis. The amount of liquid formulation the towelette deposits onto the outer surface of the skin is not so great as to leave an oily film or residue after the skin absorbs enough liquid to sufficiently moisturize the skin and thereby increase light-transmissivity through the epidermis.

All patents, patent publications and other publications cited above are incorporated herein by reference for all purposes as if expressly set forth.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1. A sealed single-use leakproof pouch comprising a material that is resistant to oily liquids, the sealed pouch containing a packaged towelette comprising an absorbent folded sheet impregnated with a liquid formulation of a metered quantity providing a consistent application of the liquid formulation over a given surface area of skin that when applied to the skin surface area, cleans and brightens the appearance of a tattoo within the skin while caring for the skin, the liquid formulation being liquid at room temperature and consisting only of botanical oils including Bergamot oil, Sunflower seed oil, Lemon oil, Rosemary Oil, and Pumpkin seed oil, the metered quantity of liquid formulation being completely absorbed by the skin surface area within one or two minutes after application to the skin surface area with no excess oil left on the skin surface area.

2-5. (canceled)

6. An article consisting of:
a single-use leakproof foil pouch, and
a folded sheet of microfiber fabric sealed within the pouch, the folded sheet impregnated with an oily fluid that is liquid at room temperature, which brightens a tattoo when rubbed over a an area of epidermal surface under which a tattoo is disposed, the oily fluid upon being absorbed by the epidermal surface area reducing light scattering of a stratum coreum layer thereby making the tattoo appear more saturated, the folded sheet impregnated with a metered quantity of the oily fluid that provides a consistent application of the oily fluid over the area of the epidural surface to be completely absorbed by the area within one or two minutes, the oily fluid consisting only of a mixture of botanical oils, including at least Bergamot oil, Sunflower seed oil, Lemon oil, Rosemary Oil, and Pumpkin seed oil.

7-11. (canceled)
